(19) United States
(12) Patent Application Publication
(10) Pub. No.: US 2020/0024636 A1
(43) Pub. Date: Jan. 23, 2020

(54) PREDICTED METABOLITES FOR BIOINDICATORS AND/OR IDENTIFICATION OF MICROBES WITH SPECIFIC METABOLITES

(71) Applicant: ExxonMobil Research and Engineering Company, Annandale, NJ (US)

(72) Inventors: Joshua T. Claypool, Cambridge, MA (US); Zarath M. Summers, High Bridge, NJ (US); Giovanni Pilloni, Jersey City, NJ (US)

(21) Appl. No.: 16/505,886

(22) Filed: Jul. 9, 2019

Related U.S. Application Data
(60) Provisional application No. 62/701,055, filed on Jul. 20, 2018.

(51) Int. Cl. C12Q 1/64; C12Q 1/6888; G16B 20/00; G16C 20/20; G16C 20/70

(52) U.S. Cl. CPC C12Q 1/64; C12Q 1/6888; C12Q 2600/158; G16C 20/20; G16C 20/70; G16B 20/00

(57) ABSTRACT

A method including: obtaining a field sample; extracting DNA from the field sample and identifying a marker gene; amplifying and sequencing the marker gene; identifying a genetic makeup of the marker gene; identifying a potential compound associated with the genetic makeup; and identifying a gene associated with the potential compound and associating that gene with a metabolite.

### CROSS-REFERENCE TO RELATED APPLICATIONS

This application claims priority to U.S. Provisional Application No. 62/701,055 filed Jul. 20, 2018, which is herein incorporated by reference in its entirety.

### FIELD

Described herein are methods for identifying organisms by one or more compounds where subsequent compounds can also serve as potential bioindicators.

### BACKGROUND

This section is intended to introduce various aspects of the art, which may be associated with exemplary embodiments of the present technological advancement. This discussion is believed to assist in providing a framework to facilitate a better understanding of particular aspects of the present technological advancement. Accordingly, it should be understood that this section should be read in this light, and not necessarily as admissions of prior art.

The exploration for and discovery of new oil reserves has become increasingly challenging and costly. Untapped reserves tend to be more difficult to identify and evaluate, and are often located subsea, which further increases the complexity and cost of discovering such reserves. Successful, efficient, and cost effective identification and evaluation of hydrocarbon-bearing reservoirs is therefore very desirable.

Currently, there is no inherent capability of any conventional tool available through literature or database searches that can identify organisms in connection to a suite of compounds that their metabolism can produce via a marker gene. Rather, scientists conduct extensive literature and database searches to trace observed compounds back to the organism(s) that produced it, and conduct unsupervised (non-targeted) discovery of bioindicators.

Large areas of metabolic bioindicator identification arises out of human-health for disease detection. For example, U.S. Patent Application 2007/0043518 describes an ensemble approach to using chemical, biochemical, and biological data to identify molecules and bioindicators. However, no known technology exists for identifying organisms with specific metabolisms to produce targeted compounds.

### SUMMARY

A method including: obtaining a field sample; extracting DNA from the field sample and identifying a marker gene; amplifying and sequencing the marker gene; identifying a genetic makeup of the marker gene; identifying a potential compound associated with the genetic makeup; and identifying a gene associated with the potential compound and associating that gene with a metabolite.

The method can further include identifying an organism, based on a relationship between the gene and the potential compound, which metabolizes a substrate of interest.

In the method, the substrate of interest can be a hydrocarbon pipeline.

In the method, the field sample can be obtained from a pig.

In the method, the potential compound can be an indole.

In the method, the potential compound can be H₂S.

The method can further include identifying a hydrocarbon reservoir based on the potential compound.

In the method, the organism can be associated with hydrocarbons.

In the method, the organism can be associated with souring or corrosion.

In the method, the organism can be associated with sulfur metabolism.

The method can further include identifying a waste stream in response to the potential compound being associated with a tailings pond, wastewater, or oil filling station.

The method can further include tracking biological diversity based on the gene.

The method can further include tracking the organism, wherein the organism degrades a hydrocarbon.

The method can further include managing hydrocarbons based on the identification of the organism.

### DESCRIPTION OF THE FIGURES

While the present disclosure is susceptible to various modifications and alternative forms, specific example embodiments thereof have been shown in the drawings and are herein described in detail. It should be understood, however, that the description herein of specific example embodiments is not intended to limit the disclosure to the particular forms disclosed herein, but on the contrary, this disclosure is to cover all modifications and equivalents as defined by the appended claims. It should also be understood that the drawings are not necessarily to scale, emphasis instead being placed upon clearly illustrating principles of exemplary embodiments of the present invention. Moreover, certain dimensions may be exaggerated to help visually convey such principles.

FIG. 1 illustrates an exemplary method for generating data for potential bioindicators or for organisms.

FIG. 2 illustrates an application of the present technological advancement for identification of compounds associated with corrosion.

FIG. 3 illustrates an application of the present technological advancement for identification of organisms capable of indole degradation.

FIG. 4 illustrates an application of the present technological advancement for identification of organisms responsible for hydrogen sulfide production and subsequently related to microbial souring and corrosion.

FIG. 5 illustrates an exemplary computer system that can execute aspects of the present technological advancement.

### DETAILED DESCRIPTION

Exemplary embodiments are described herein. However, to the extent that the following description is specific to a particular embodiment, this is intended to be for exemplary purposes only and simply provides a description of examples of the present technological advancement. Accordingly, the invention is not limited to the specific embodiments described below, but rather, it includes all alternatives, modifications, and equivalents falling within the true spirit and scope of the appended claims.

### Overview

Described herein are methods for identifying organisms by one or more compounds where subsequent compounds can also serve as potential bioindicators. Currently, predicted metagenomes only explore the genes that the organisms may have, and it is left up to researchers to identify what compounds the organisms may use as substrates or produce as an intermediate or end product. As many genes are connected directly to compounds that they are responsible for acting upon, this will produce a connection between the predicted metagenomic host of the gene and the compound. The knowledge of the compounds that can be derived from a community of organisms can be used to either identify organisms with a metabolism of interest or to produce a list of compounds that could serve as potential bioindicators.

The present technological advancement has several applications in the oil and gas industry. In the hydrocarbon exploration context, the present technological advancement can identify bioindicators associated with reservoirs and locating organisms associated with (i.e., as an indicator of the presence of) subsurface hydrocarbon deposits. The subsurface hydrocarbon deposit may be further characterized by the type of hydrocarbon-related genes that can provide context regarding hydrocarbon qualities such as API. In the hydrocarbon development context, the present technological advancement can improve well location through bioindicator screening. By tracing the genetic potential of microbial hydrocarbon metabolism through soil core samples, improved placement of wells can be accomplished. In the hydrocarbon production context, the present technological advancement can classify compounds associated with souring or corrosion, and characterize organisms involved with sulfur metabolism for development of mitigation strategies. This may result in the use of a microbial genetic indicator that can serve as an early warning for corroding pipelines prior to failure. In an environmental context, the present technological advancement can identify rogue waste streams by correlation with waste-associated compounds (tailings ponds, wastewater, oil filling stations, etc.), track biological diversity to maintain organisms connected to environmental substrates such as to not affect the accumulation or loss of critical organisms, and discover organisms to degrade crude-oil, gasoline, and other petrol-derived compounds that may have been released into the environment. In a biofuels context, the present technological advancement can provide information relevant to the deconstruction of feedstock, production of energy-related molecules and conversion of biological inhibitors. In the biochemical context, the present technological advancement can upgrade molecules from hydrocarbons. The present technological advancement can also identify a set of control compounds that can serve to identify fouling, clogging, or non-planktonic growth (biofilms) in difficult to sample area.

While the preceding provides an non-exhaustive list of benefits that the present technological advancement can bring to the oil and gas industry, the discussed benefits are not necessarily limited to any particular activity and those of ordinary skill in the art will recognize that benefits to one activity (e.g., exploration) are also present in the others (e.g., development and production).

Conventionally, identifying organisms and/or a potential set of bioindicators requires time and highly skilled personnel to narrow the number and/or types of organisms/compounds being studied. The present technological advancement can provide a streamlined approach to identify a probable set of organism/compound relationships that could be created from a community of organisms.

Identifying biogenic activity has tremendous value across many industries, such as oil and gas, agriculture, and human health. While the extraction and sequencing of DNA has been a revolutionary tool to advance the detection of biogenic activity, sifting through data, literature, and databases to derive critical information about organismal metabolism is still the norm. Streamlining this process can facilitate discoveries of organisms with unique metabolisms or to identify bioindicators that can be monitored. More time from the highly skilled work can be put into analysis instead of literature searches.

### Definitions

Various terms as used herein are defined below. To the extent a term used in a claim is not defined below, it should be given the broadest possible definition persons in the pertinent art have given that term as reflected in at least one printed publication or issued patent.

The term “and/or” as used in a phrase such as “A and/or B” herein is intended to include “A and B”, “A or B”, “A”, and “B”.

“Amplification” is the generation of multiple copies of nucleic acid segments to enhance the analysis of very low amounts of nucleic acids. For example, amplification may be performed by a polymerase chain reaction (“PCR”), which uses a thermostable polymerase enzyme, such as the TAQ enzyme for DNA, to exponentially produce thousands or millions of copies of a DNA segment during a number of thermal cycles. During each cycle, the DNA segments produced in a previous cycle become templates for new copies of that segment. RNA analysis may be performed by reverse transcription of the RNA to create cDNA segments, which may then be amplified.

As used herein, “DNA analysis” refers to any technique used to amplify and/or sequence DNA contained within the sample. DNA amplification can be accomplished using PCR techniques. DNA analysis may also comprise non-targeted, non-PCR based DNA sequencing (e.g., metagenomics) techniques. As a non-limiting example, DNA analysis may include sequencing the hyper-variable region of the 16S rDNA (ribosomal DNA) and using the sequencing for species identification via DNA.

The term “field sample” refers to a sample containing material from the natural environment. Field samples include, but are not limited to, samples taken from any soil (encompassing all soil types and depths), water or liquid (encompassing freshwater aquatic or marine habitats), sediment (encompassing marine sediment, lake or river sediment, or mud sediment), or atmospheric dust or particulates. The field sample may include a multitude of species of microorganisms or a single species of microorganism. In preferred embodiments, the samples are field samples taken from the sediment or water column near a hydrocarbon seep or water near a submerged pipeline used for hydrocarbons transport. In such a context, the term “near” means the sample is obtained within a radius of 150 meters, or 125 meters, or 100 meters, or 75 meters, or 50 meters, or 25 meters, or 20 meters, or 15 meters, or 10 meters, or 5 meters, or 3 meters, or 1 meter from the center of the location where the seep is emanating from the surface or from any given location along the pipeline length. Reference samples may also be field samples taken from the sediment or water column away from the hydrocarbon seep or away from the pipeline. In such a context, the term “away” means the reference sample is obtained at least 200 meters, or at least 250 meters, or at least 300 meters, or at least 350 meters, or at least 400 meters, or at least 450 meters, or at least 500 meters away from the center of the location where the seep is emanating from the surface, and in some embodiments, less than 2000 meters, or less than 1750 meters, or less than 1500 meters, or less than 1250 meters, or less than 1000 meters away from the location where the seep is emanating from the surface. Similarly, for pipelines the term “away” means the reference sample is obtained at least 200 meters, or at least 250 meters, or at least 300 meters, or at least 350 meters, or at least 400 meters, or at least 450 meters, or at least 500 meters away from the location of the pipeline, and in some embodiments, less than 2000 meters, or less than 1750 meters, or less than 1500 meters, or less than 1250 meters, or less than 1000 meters away from the location where the pipeline is located. Field samples can be manually obtained or obtained via automatic sample collection and/or analysis.

“Automatic sample collection and/or analysis” is, especially for pipeline, one or more devices that can independently and continuously inspect and/or analyze the water close to the pipeline external wall or other structure and communicate the results in real time or near real time.

As used herein, “genomics” refers to the study of genomes of organisms, which includes the determination of the entire DNA or RNA sequence of organisms as well as genetic mapping.

A “hydrocarbon” is an organic compound that primarily includes the elements hydrogen and carbon, although nitrogen, sulfur, oxygen, metals, or any number of other elements may also be present in small amounts. As used herein, hydrocarbons generally refer to organic materials (e.g., natural gas and liquid petroleum) that are harvested from hydrocarbon containing sub-surface rock layers, termed reservoirs.

As used herein, “hydrocarbon management” or “managing hydrocarbons” includes hydrocarbon exploration, hydrocarbon development, hydrocarbon extraction, hydrocarbon production, identifying potential hydrocarbon resources, identifying well locations, determining well injection and/or extraction rates, identifying reservoir connectivity, acquiring, disposing of and/or abandoning hydrocarbon resources, reviewing prior hydrocarbon management decisions, and any other hydrocarbon-related acts or activities.

As used herein, “hydrocarbon exploration” refers to any activity associated with determining the location of hydrocarbons in subsurface regions. Hydrocarbon exploration normally refers to any activity conducted to obtain measurements through acquisition of measured data associated with the subsurface formation and the associated modeling of the data to identify potential locations of hydrocarbon accumulations. Accordingly, hydrocarbon exploration includes acquiring measurement data, modeling of the measurement data to form subsurface models, and determining the likely locations for hydrocarbon reservoirs within the subsurface. The measurement data may include seismic data, gravity data, magnetic data, electromagnetic data, and the like.

As used herein, “hydrocarbon development” refers to any activity associated with planning of extraction and/or access to hydrocarbons in subsurface regions. Hydrocarbon development normally refers to any activity conducted to plan for access to and/or for production of hydrocarbons from the subsurface formation and the associated modeling of the data to identify preferred development approaches and methods. By way of example, hydrocarbon development may include modeling of the subsurface formation and extraction planning for periods of production, determining and planning equipment to be utilized and techniques to be utilized in extracting the hydrocarbons from the subsurface formation, and the like.

As used herein, “hydrocarbon production” refers to any activity associated with extracting hydrocarbons from subsurface location, such as a well or other opening. Hydrocarbon production normally refers to any activity conducted to form the wellbore along with any activity in or on the well after the well is completed. Accordingly, hydrocarbon production or extraction includes not only primary hydrocarbon extraction, but also secondary and tertiary production techniques, such as the injection of gas or liquid for increasing drive pressure, mobilizing the hydrocarbon, or treating the well by, for example, chemicals, or hydraulic fracturing the wellbore to promote increased flow, well servicing, well logging, and other well and wellbore treatments.

As used herein, “metabolites” refer to compounds produced by bacteria and/or archaea during respiration or fermentation. For example, acetic acid is an example of a metabolite. Metabolites can provide information about the type of hydrocarbon being used as a substrate as well as information about physical and chemical conditions in the reservoirs. For example, the presence of specific metabolites may indicate or infer the presence of hydrocarbons and/or conditions at depth.

A “microbe” is any microorganism that is of the domain Bacteria, Eukarya, or Archaea. Microbes include bacteria, fungi, nematodes, protozoans, archaebacteria, algae, dinoflagellates, molds, bacteriophages, mycoplasma, viruses, and viroids.

The term “nucleic acid” refers to biopolymers used in cells for the transfer of information. Nucleic acids include deoxyribonucleic acid (“DNA”), which is generally found in a nucleus of a eukaryotic cell, and ribonucleic acid (“RNA”), which is generally found in the cytoplasm of a eukaryotic cell. A prokaryotic cell, such as a bacterial or archaeal cell, does not have a nucleus, and both DNA and RNA may be found in the cytoplasm of the cell. DNA often provides the genetic code for a cell, although a few types of organisms use RNA to carry heritable characteristics. RNA is often associated with the synthesis of proteins from genes on the DNA.

As used herein, “products” refer to proteins, lipids, exopolymeric substances, and other cellular components that organisms produce under a given set of conditions.

As used herein, “RNA analysis” refers to any technique used to amplify and/or sequence RNA contained within the samples. The same techniques used to analyze DNA can be used to amplify and sequence RNA. RNA, which is less stable than DNA is the translation of DNA in response to a stimuli. Therefore, RNA analysis may provide a more accurate picture of the metabolically active members of the community and may be used to provide information about the community function of organisms in a sample.

A “reservoir” is a subsurface rock formation from which a production fluid can be produced. The rock formation may include granite, silica, carbonates, clays, and organic matter, such as oil, gas, or coal, among others. Reservoirs can vary in size from less than one cubic foot (0.3048 m³) to hundreds of cubic feet (hundreds of cubic meters). The permeability of the reservoir rock may provide paths for production and for hydrocarbons to escape from the reservoir and move to the surface.

A “pig” is a device used to clean pipelines and/or displace hydrocarbons from pipelines.

A “pipeline” is a tube or system of tubes used for transporting crude oil and natural gas.

As used herein, “sequencing” refers to the determination of the exact order of nucleotide bases in a strand of DNA (deoxyribonucleic acid) or RNA (ribonucleic acid) or the exact order of amino acids residues or peptides in a protein. For example, nucleic acid sequencing can be done using Sanger sequencing or next-generation high-throughput sequencing including but not limited to massively parallel pyrosequencing, Illumina sequencing, or SOLiD sequencing, ion semiconductor sequencing. For example, amino acid sequencing may be done by mass spectrometry and Edman degradation.

“Substantial” when used in reference to a quantity or amount of a material, or a specific characteristic thereof, refers to an amount that is sufficient to provide an effect that the material or characteristic was intended to provide. The exact degree of deviation allowable may in some cases depend on the specific context.

### Exemplary Embodiments

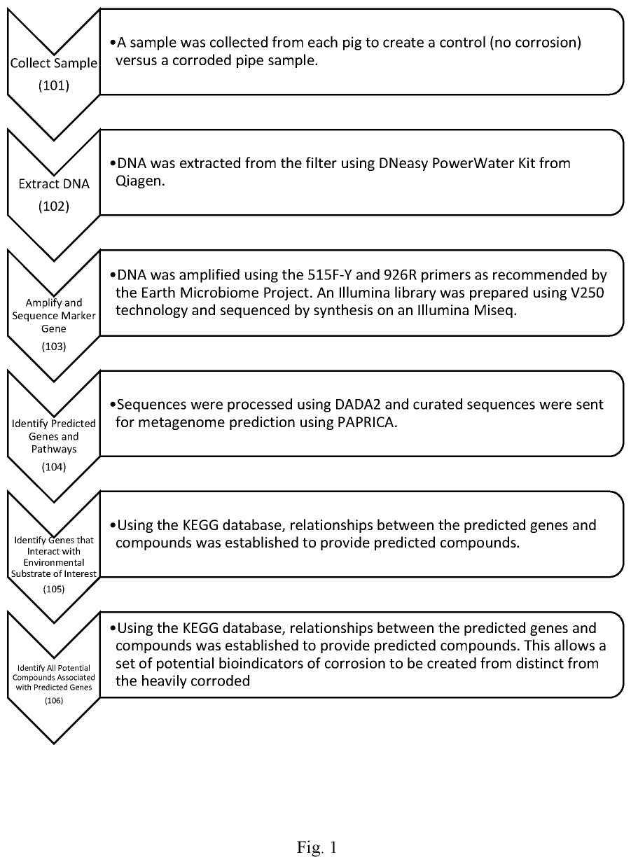

FIG. 1 illustrates an exemplary method for generating data for potential bioindicators or for organisms.

Step 101 includes collecting a field sample. The field sample can be collected with any techniques that preserves the integrity of the sample (e.g., a pig). When field samples are collected via automated machines, those of ordinary skill in the art will appreciate that such machine can be programmed and equipped in order to process the samples. Thus, at least some of the following steps could be conducted with automatic sample collection and/or analysis.

Step 102 includes extracting DNA from the field sample. Any known DNA extraction technique can be used. For example, nucleic acids (e.g., DNA and RNA) and proteins are extracted from the sample. Proteins can be extracted from the sample and purified using known techniques. For example, the proteins can be separated from the sample using two dimensional electrophoresis or standard precipitation techniques. The nucleic acids can be extracted from the sample using known techniques. For example, nucleic acids can be extracted from a sediment sample using a sediment DNA extraction technique, such as the MoBio Power Soil DNA extraction kit, or utilizing the method described in U.S. patent application Ser. No. 15/600,161, the disclosure of which is incorporated herein by reference.

Step 103 includes amplifying and sequencing a marker gene. Marker genes are genes that allow identification of an organism without sequencing an entire organism. Various marker genes exist for various walks of life. A thorough but not comprehensive list of these marker genes is: 16S rRNA, Internal Transcribed Spacer (ITS), 18S rRNA, mcrA, pmoA, amoA, rpoB, nirS, nirK, nosZ, pufM [2, 3]. Marker gene selection is usually made in coordination with the type or types of organisms being sequenced. Upon selection of the appropriate marker gene, the gene or gene fragment is amplified using polymerase chain reactions (PCR) [4]. These amplified sequences can then be sequenced using, but not limited to, Sanger, Roches 454, Illumina Sequencing-by-Synthesis, Oxford Technologies IonTorrent, and PacBio Single Molecule Real-Time. The goal of sequencing via any of these technologies or those yet to be developed is to produce a set of genomic character sequences that are representative of the marker genes amplified prior to sequencing. Many sequencing technologies produce small amounts of error in their process and as a result post-processing of the genome character sequences must occur, usually in-silico due to the high volumes. Such available programs to handle the in-silico post-processing are but not limited to, DADA2, deblur, and VSEARCH [5-7]. The outcome of this step is to arrive at a set of quality-controlled character sequences that are representative of the organism(s) that are present in a sample.

Step 104 includes identifying predicted genes and metabolic pathways. Pathways are referring to metabolic pathways from which the organism may use to generate energy, balance cell metabolites associated with reduction and oxidation, and to respond to external stimuli. Identification of predicted genes and/or metabolic pathways has become a valuable tool for the study of communities using marker gene amplification methods. Tools such as Tax4Fun, PAPRICA, and PICRUSt have utilized different methods to arrive at a final prediction of genes and pathways for the collection of organisms identified within a sample [8-10]. These tools are capable of generating a prediction for organisms that have yet to be discovered or identified. These tools work by connecting the unique sequences from the previous step to their respective hypothetical genomes/collection of genes. These genes are then collapsed into their respective pathway/set of metabolic reactions to serve as a grouping mechanism for the collective function of genes. The genes and pathways that these tools utilize for prediction are derived from but not limited to public and private databases such as BioCyc, MetaCyc, Kyoto Encyclopedia of Genes and Genomes (KEGG), EggNOG, NCBI [11-15]. The outcome is a set of counts of the number of occurrences of unique genes and/or pathways associated with the entire collection of organisms identified from marker gene sequencing.

Step 105 includes identifying genes associated with the potential compounds. Every metabolic reaction uses a gene or set of genes to convert one chemical to another. These chemicals are usually referred to as metabolites in biology as there are produced as a result of some metabolic reaction. Through the use of the public and private databases, BioCyc, KEGG, et cetera, the connection between genes and the metabolites they interact with can be collected [12, 15]. This would provide an initial scope of all potential metabolites/chemicals that can be selectively monitored in place of a community. For applications interested in identifying bioindicators, the method of FIG. 1 can end at step 105. Bioindicators are organisms that are unambiguously found in specific environments with defined characteristic. Bioindicators in this case are being referring to as measurable metabolites that are a result of microbial metabolism and are indicative of a characteristic state. Examples of a characteristic state would be: sick/not-sick, soil quality, oil quality. However, for applications interested in identifying organisms, the method can continue to step 106.

Step 106 includes identifying organism(s) capable of metabolizing an environmental substrate of interest. An environmental substrate of interest is a molecule that can be produced or consumed by a microbe that has been previously identified as having relevance to a process, characteristic state, or otherwise relevant (i.e., the microbe is a bioindicator). This step can include connecting the original marker gene that was sequenced to the gene and eventually compound such that a reverse lookup can be generated to identify organisms of interest. Once genes have been connected to their chemical/metabolites that they interact with, the connection can be shortened to the marker gene sequence in the sample that also connects to that chemical/metabolite. As it is often desirable to know which organisms are interacting with a particular chemical, this provides a systematic process at which one can arrive at these conclusions.

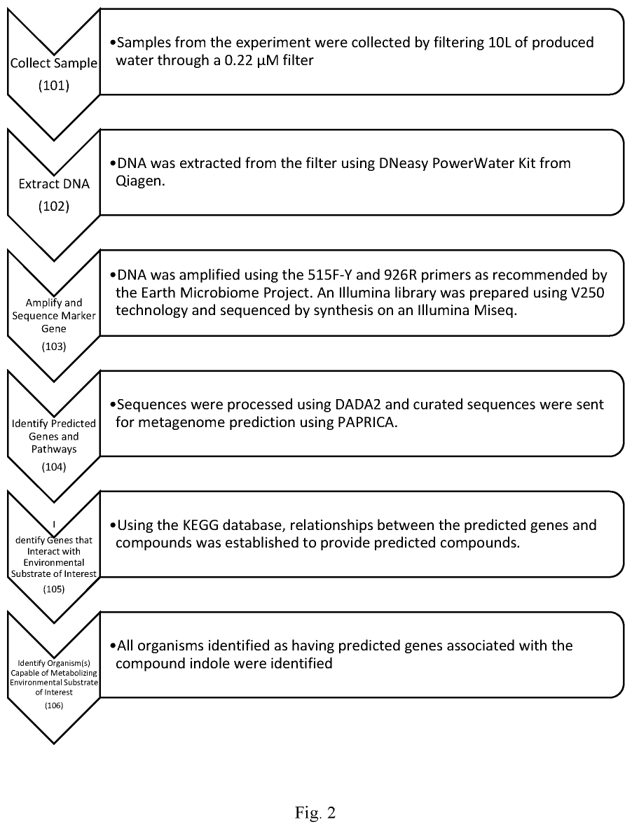

FIG. 2 illustrates an application of the present technological advancement for identification of compounds associated with corrosion. FIG. 2 illustrates how the steps of FIG. 1 were applied to hypothetical case study. Microbial corrosion can be an expensive problem where the costs are associated with premature replacement of pipelines, pipeline cleaning, and environmental cleanup from leaks. Identification of corrosion at early stages can help reduce costs, but many organisms associated with corrosion may not appear in produced water because of their growth as biofilms. Thereby, having a molecular bioindicator associated uniquely with biofilm forming-corrosion-causing-microbes, would improve detection of corrosion causing organisms.

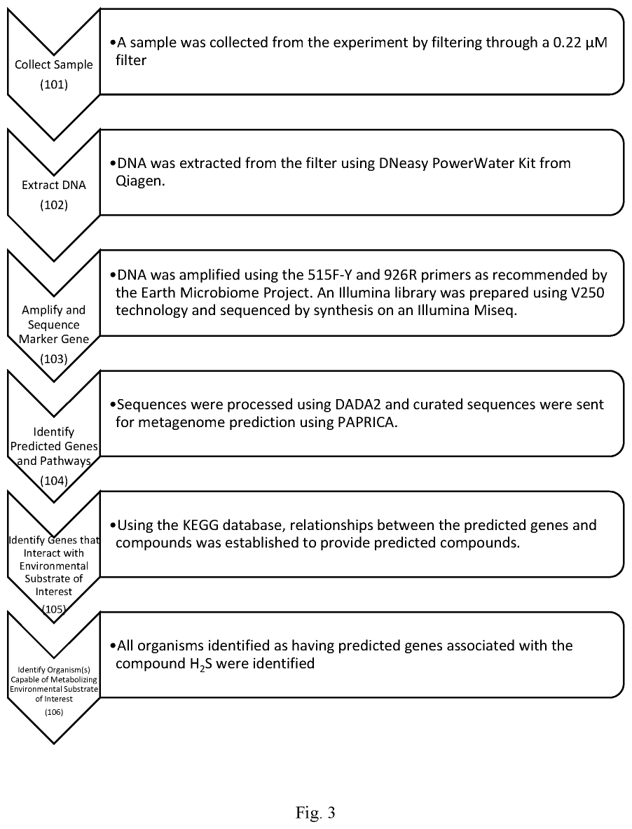

FIG. 3 illustrates an application of the present technological advancement for identification of organisms capable of indole degradation. FIG. 3 illustrates how the steps of FIG. 1 were applied to hypothetical case study. Indole and its derivatives will adversely affect the catalyst associated with cracking of crude oil and it is expensive to remove. Therefore, the use of biology for tiodenitrogenation′, the biological removal of nitrogen from mono or polycyclic aromatic compounds, has become of increasing interest. However, the organisms surrounding indole degradation are not well-characterized [1]. It would therefore be of interest to identify these organisms that may harbor the enzymes associated with indole degradation. Therefore an experiment could be carried out in a lab to enrich microorganisms capable by subjecting a source of microorganisms to indole and its derivatives at various temperatures.

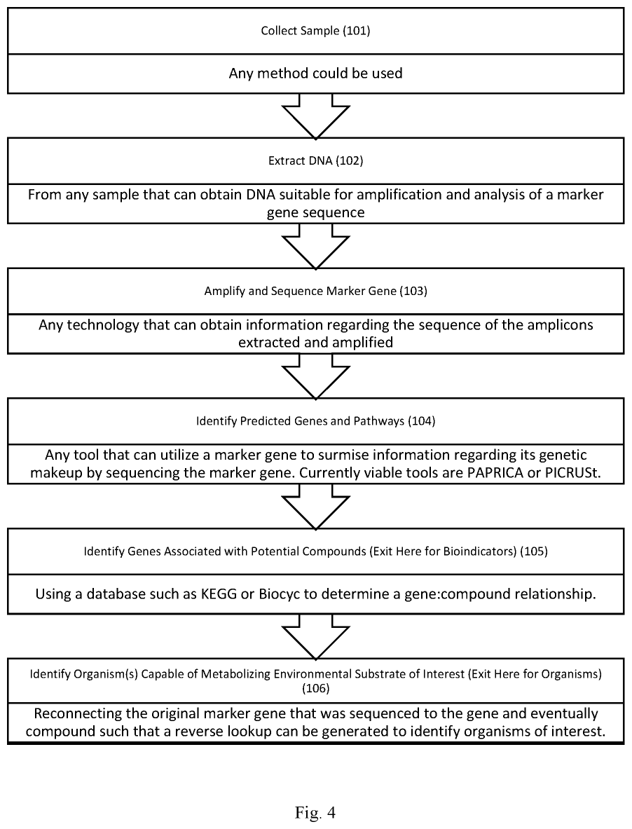

FIG. 4 illustrates an application of the present technological advancement for identification of organisms responsible for hydrogen sulfide production and subsequently related to microbial souring and corrosion. Hydrogen sulfide (H₂S) is a large problem in oil and gas because of the damage it does to infrastructure and the regulatory requirements for removal of sulfur compounds. Current work in this area has targeted organisms with a potential sulfur metabolism but has not discriminated sulfur metabolism from hydrogen sulfide metabolism. Thereby a site that is experiencing large amounts of corrosion would be selected for field sampling.

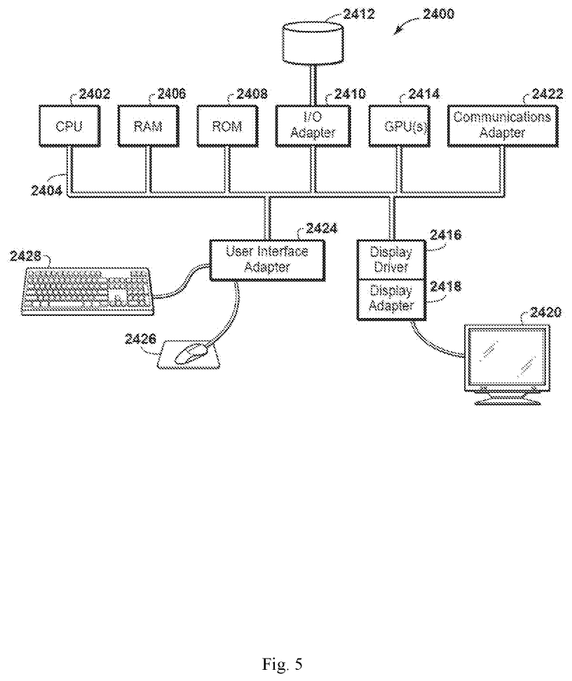

FIG. 5 is a block diagram of a computer system 2400 that can be used to execute control the methods and systems discussed herein. Those of ordinary skill in the art will recognize that such a computer system can be interfaced with conventional equipment used to perform the various analyzes described above. A central processing unit (CPU) 2402 is coupled to system bus 2404. The CPU 2402 may be any general-purpose CPU, although other types of architectures of CPU 2402 (or other components of exemplary system 2400) may be used as long as CPU 2402 (and other components of system 2400) supports the operations as described herein. Those of ordinary skill in the art will appreciate that, while only a single CPU 2402 is shown in FIG. 5, additional CPUs may be present. Moreover, the computer system 2400 may comprise a networked, multi-processor computer system that may include a hybrid parallel CPU/GPU system. The CPU 402 may execute the various logical instructions according to various teachings disclosed herein. For example, the CPU 2402 may execute machine-level instructions for performing processing according to the operational flow described.

The computer system 2400 may also include computer components such as nontransitory, computer-readable media. Examples of computer-readable media include a random access memory (RAM) 2406, which may be SRAM, DRAM, SDRAM, or the like. The computer system 2400 may also include additional non-transitory, computer-readable media such as a read-only memory (ROM) 2408, which may be PROM, EPROM, EEPROM, or the like. RAM 2406 and ROM 2408 hold user and system data and programs, as is known in the art. The computer system 2400 may also include an input/output (I/O) adapter 2410, a communications adapter 2422, a user interface adapter 2424, and a display adapter 2418.

The I/O adapter 2410 may connect additional non-transitory, computer-readable media such as a storage device(s) 2412, including, for example, a hard drive, a compact disc (CD) drive, a floppy disk drive, a tape drive, and the like to computer system 2400. The storage device(s) may be used when RAM 2406 is insufficient for the memory requirements associated with storing data for operations of the present techniques. The data storage of the computer system 2400 may be used for storing information and/or other data used or generated as disclosed herein. For example, storage device(s) 2412 may be used to store configuration information or additional plug-ins in accordance with the present techniques. Further, user interface adapter 2424 couples user input devices, such as a keyboard 2428, a pointing device 2426 and/or output devices to the computer system 400. The display adapter 2418 is driven by the CPU 2402 to control the display on a display device 2420 to, for example, present information to the user regarding available plug-ins.

The architecture of system 2400 may be varied as desired. For example, any suitable processor-based device may be used, including without limitation personal computers, laptop computers, computer workstations, and multi-processor servers. Moreover, the present technological advancement may be implemented on application specific integrated circuits (ASICs) or very large scale integrated (VLSI) circuits. In fact, persons of ordinary skill in the art may use any number of suitable hardware structures capable of executing logical operations according to the present technological advancement. The term “processing circuit” encompasses a hardware processor (such as those found in the hardware devices noted above), ASICs, and VLSI circuits. Input data to the computer system 2400 may include various plug-ins and library files. Input data may additionally include configuration information.

### CONCLUSION

The present techniques may be susceptible to various modifications and alternative forms, and the examples discussed above have been shown only by way of example. However, the present techniques are not intended to be limited to the particular examples disclosed herein. Indeed, the present techniques include all alternatives, modifications, and equivalents falling within the spirit and scope of the appended claims.

### REFERENCES

The following documents numbered 1 through 16 are hereby incorporated by reference in their entirety.
1. Bachmann, R. T., A. C. Johnson, and R. G. J. Edyvean, Biotechnology in the petroleum industry: An overview. International Biodeterioration & Biodegradation, 2014. 86 (Part C): p. 225-237.
2. Lan, Y., G. Rosen, and R. Hershberg, Marker genes that are less conserved in their sequences are useful for predicting genome-wide similarity levels between closely related prokaryotic strains. Microbiome, 2016. 4(1): p. 18.
3. Knight, R., et al., Best practices for analysing microbiomes. Nature Reviews Microbiology, 2018.
4. Clark, D. P. and N. J. Pazdernik, Chapter e6 —Polymerase Chain Reaction, in Molecular Biology (Second Edition). 2013, Academic Press: Boston. p. e55-e61.
5. Rognes, T., et al., VSEARCH: a versatile open source tool for metagenomics. PeerJ, 2016. 4: p. e2584.
6. Amir, A., et al., Deblur Rapidly Resolves Single-Nucleotide Community Sequence Patterns. mSystems, 2017. 2(2).
7. Callahan, B. J., et al., DADA2: High-resolution sample inference from Illumina amplicon data. Nature Methods, 2016. 13: p. 581.
8. Ashauer, K. P., et al., Tax4Fun: predicting functional profiles from metagenomic 16S rRNA data. Bioinformatics, 2015. 31(17): p. 2882-2884.
9. Bowman, J. S. and H. W. Ducklow, Microbial Communities Can Be Described by Metabolic Structure: A General Framework and Application to a Seasonally Variable, Depth-Stratified Microbial Community from the Coastal West Antarctic Peninsula. PLOS ONE, 2015. 10(8): p. e0135868.
10. Langille, M. G. I., et al., Predictive functional profiling of microbial communities using 16S rRNA marker gene sequences. Nat Biotech, 2013. 31(9): p. 814-821.
11. Pruitt, K. D., T. Tatusova, and D. R. Maglott, NCBI reference sequences (RefSeq): a curated non-redundant sequence database of genomes, transcripts and proteins. Nucleic Acids Research, 2007. 35(suppl_1): p. D61-D65.
12. Kanehisa, M. and S. Goto, KEGG: Kyoto Encyclopedia of Genes and Genomes. Nucleic Acids Research, 2000. 28(1): p. 27-30.
13. Caspi, R., et al., MetaCyc: a multiorganism database of metabolic pathways and enzymes. Nucleic Acids Research, 2006. 34(Database issue): p. D511-D516.
14. Huerta-Cepas, J., et al., eggNOG 4.5: a hierarchical orthology framework with improved functional annotations for eukaryotic, prokaryotic and viral sequences. Nucleic Acids Research, 2016. 44(D1): p. D286-D293.
15. Karp, P. D., et al., Expansion of the BioCyc collection of pathway/genome databases to 160 genomes. Nucleic Acids Research, 2005. 33(19): p. 6083-6089.
16. U.S. Patent Application 2007/0043518.

1. A method comprising:
obtaining a field sample;
extracting DNA from the field sample and identifying a marker gene;
amplifying and sequencing the marker gene;
identifying a genetic makeup of the marker gene;
identifying a potential compound associated with the genetic makeup; and
identifying a gene associated with the potential compound and associating that gene with a metabolite.

2. The method of claim 1, further comprising identifying an organism, based on a relationship between the gene and the potential compound, which metabolizes a substrate of interest.

3. The method of claim 1, wherein the substrate of interest is a hydrocarbon pipeline.

4. The method of claim 1, wherein the field sample was obtained from a pig.

5. The method of claim 1, wherein the potential compound is indole.

6. The method of claim 1, wherein the potential compound is H₂S.

7. The method of claim 1, further comprising identifying a hydrocarbon reservoir based on the potential compound.

8. The method of claim 2, wherein the organism is associated with hydrocarbons.

9. The method of claim 6, wherein the organism is associated with souring or corrosion.

10. The method of claim 2, wherein the organism is associated with sulfur metabolism.

11. The method of claim 1, further comprising identifying a waste stream in response to the potential compound being associated with a tailings pond, wastewater, or oil filling station.

12. The method of claim 1, further comprising tracking biological diversity based on the gene.

13. The method of claim 2, further comprising tracking the organism, wherein the organism degrades a hydrocarbon.

14. The method of claim 2, further comprising managing hydrocarbons based on the identification of the organism.
